(19) United States
(12) Patent Application Publication
(10) Pub. No.: US 2022/0196666 A1
(43) Pub. Date: Jun. 23, 2022

(54) Graphene-Based Sensor For Detection Of Prostate Biomarkers

(71) Applicant: GRAPHENE-DX, INC., Boston, MA (US)

(72) Inventors: Mehdi Abedi, Brighton, MA (US); Namal Nawana, Weston, MA (US); Mohammad E. Taslim, Needham, MA (US); Mohammed Fotouhi, Weston, MA (US); Edward A. Greenfield, Stoughton, MA (US)

(21) Appl. No.: 17/542,400

(22) Filed: Dec. 4, 2021

Related U.S. Application Data
(60) Provisional application No. 63/122,281, filed on Dec. 7, 2020.

(51) Int. Cl. G01N 33/574; G01N 27/12

(52) U.S. Cl. CPC G01N 33/57434; G01N 2333/96455; G01N 27/125

(57) ABSTRACT

The present teachings are generally directed to sensors that employ antibody- and/or aptamer-functionalized graphene layer (or graphene flakes and/or graphdiyne layer) for detecting a prostate-specific biomarker in a sample. A graphene layer can be deposited on a underlying substrate and functionalized with an antibody and/or aptamer that specifically binds with an analyte of interest (e.g., a prostate-specific biomarker). A sample under investigation can be introduced onto the functionalized graphene layer. The interaction of the analyte of interest, if present in the sample, with the functionalized graphene layer can mediate a change in at least one electrical property of the graphene layer, e.g., their DC electrical resistance. An analyzer can detect such a change and analyze it to determine whether the analyte is present in the sample. In some embodiments, calibration methods can be employed to quantify the analyte present in the sample.

### RELATED APPLICATION

This application claims priority to U.S. Provisional Patent Application No. 63/122,281, filed Dec. 7, 2020, entitled “Graphene-Based Sensor for Detection of Prostate Biomarkers,” which is hereby incorporated by reference in its entirety.

### TECHNICAL FIELD

The present disclosure relates generally to systems and methods for detecting and/or quantifying, biomarkers specific to prostate based on antibody- and/or aptamer-functionalized allotropes of carbon, such as graphene, graphdiyne, and the like.

### BACKGROUND

Prostate-specific antigen (PSA) is a protein produced by cells of the prostate gland and can be a biomarker for prostate cancer. The conventional methods of blood testing for PSA can be time-consuming and/or can require complex sample preparation.

Accordingly, there is a need for improved systems and methods for detecting prostate-specific biomarkers.

### SUMMARY

In one aspect, a sensor for detecting a prostate-specific bio-marker in a sample is disclosed, which comprises a graphene layer deposited on an underlying substrate, a plurality of antibodies and/or aptamers coupled to the graphene layer to generate an antibody- and/or an aptamer-functionalized graphene layer, wherein the antibodies and/or aptamers exhibit specific binding to at least one prostate-specific biomarker, and a plurality of electrical conductors electrically coupled to the graphene layer for measuring an electrical property thereof.

In some embodiments, the prostate-specific biomarker can be a prostate-specific antigen (PSA). In other embodiments, the prostate-specific biomarker can be kallikrein-related peptidase 2, prostate cancer antigen 3 (PCA3), TMPRSS2-ERG gene fusion, or the like.

In some embodiments, the sensor can include a reference electrode that is disposed n proximity of the functionalized graphene layer. An AC voltage source can be used to apply an AC voltage to the reference electrode. In some embodiments, the AC voltage source can be programmed to apply an AC voltage with a frequency in a range of about 1 kHz to about 1 MHz to the reference electrode. In some embodiments, the amplitude of the applied AC voltage can be in a range of about 100 millivolts to about 3 volts. In some embodiments, in addition to the AC voltage, a DC ramp voltage can be applied to the reference electrode. By way of example, in some such embodiment, the DC ramp voltage can vary between −40 volts to +40 volts.

In some embodiments, the measured electrical property of the functionalized graphene layer can be related to a change in the electron mobility within the functionalized graphene layer. For example, in some embodiments, the measured electrical property of the functionalized graphene layer can be its DC electrical resistance, which can change in response to specific binding of a target analyte to the antibodies and/or aptamers. By way of example, a detected change in the mobility of electrons in the functionalized graphene layer in response to specific binding of a target analyte to the antibodies and/or aptamers can be employed to detect the target analyte.

The graphene layer can be deposited on a variety of different substrates. By way of example, in some embodiments, the substrate can be a semiconductor substrate, such as silicon. In other embodiments, the substrate can be a glass substrate. In yet other embodiments, the substrate can be a polymeric substrate (e.g., a plastic substrate).

In a related aspect, a method of detecting a prostate-specific biomarker in a sample is disclosed, which comprises applying the sample to a graphene layer that is functionalized with an antibody and/or an aptamer exhibiting specific binding to the prostate-specific biomarker, measuring at least one electrical property of the functionalized graphene layer, and using the measured electrical property to determine whether the prostate-specific biomarker is present in the sample.

In some embodiments, the method can further include quantifying the prostate-specific biomarker in the sample. By way of example, the sensor can be calibrated to allow the quantification of the detected prostate-specific biomarker.

In another aspect, a sensor for detecting a prostate-specific biomarker in a sample is disclosed, which comprises a graphdiyne layer deposited on an underlying substrate, a plurality of antibodies and/or aptamers coupled to the graphdiyne layer to generate functionalized graphdiyne layer (e.g., an antibody- and/or an aptamer-functionalized graphdiyne layer), wherein the antibodies and/or aptamers exhibit specific binding to the prostate-specific biomarker, and a plurality of electrical conductors electrically coupled to the graphdiyne layer for measuring an electrical property thereof. In some embodiments, the graphdiyne layer can comprise a plurality of graphdiyne flakes.

In other embodiments, rather than employing a graphene or a graphdiyne layer, a plurality of graphene flakes functionalized with one or more antibodies and/or aptamers can be employed.

In any of the above embodiments, rather than employing a single type of antibody and/or aptamer, a plurality of different types of antibodies and/or aptamers that exhibit specific binding to different prostate-specific biomarkers can be employed. By way of example, a graphene layer of a sensor according to the present teachings can be functionalized with a plurality of antibodies and/or aptamers, where some of the antibodies and/or aptamers exhibit specific binding to one prostate-specific biomarker and other antibodies and/or aptamers exhibit specific binding to a different prostate-specific biomarker.

The systems and methods according to the present teachings can be employed to investigate a variety of different samples, such as biological samples including blood, urine, semen, among others.

Note that the various embodiments described above can be combined with any other embodiments described herein. The features and advantages described in the specification are not all inclusive and, in particular, many additional features and advantages will be apparent to one of ordinary skill in the art in view of the drawings, specification, and claims. Moreover, it should be noted that the language used in the specification has been principally selected for readability and instructional purposes, and may not have been selected to delineate or circumscribe the inventive subject matter.

### BRIEF DESCRIPTION OF THE DRAWINGS

So that the present disclosure can be understood in greater detail, a more particular description may be had by reference to the features of various embodiments, some of which are illustrated in the appended drawings. The appended drawings, however, merely illustrate pertinent features of the present disclosure and are therefore not to be considered limiting, for the description may admit to other effective features.

FIG. 1 schematically depicts a sensor according to an embodiment, which includes a plurality of graphene nano-flakes deposited over a substrate, in accordance with some embodiments.

FIG. 2 schematically depicts an analyzer suitable for measuring electrical resistance of the sensor depicted in FIG. 1, in accordance with some embodiments.

FIG. 3 schematically depicts a device for measuring the electrical resistance of the sensor depicted in FIG. 1, in accordance with some embodiments.

FIG. 4A schematically depicts a sensor according to an embodiment, which includes a reference electrode positioned in proximity of the graphene nano-flakes, in accordance with some embodiments.

FIG. 4B schematically depicts a combination of a ramp voltage and an AC voltage applied to the reference electrode of the sensor, in accordance with some embodiments.

FIGS. 5A and 5B schematically depict a sensor according to an embodiment suitable for detecting an analyte in a sample, in accordance with some embodiments.

FIG. 6 schematically depicts an embodiment of the sensor depicted in FIGS. 5A and 5B in which a reference electrode is positioned in proximity of the functionalized graphene nano-flakes, in accordance with some embodiments.

FIG. 7 schematically depicts a sensor according to an embodiment, which can be used to detect prostate-specific biomarker in a sample, in accordance with some embodiments.

FIG. 8 schematically depicts a graphene layer according to an embodiment, which comprises a plurality of graphene and/or graphdiyne flakes deposited on a graphene or graphdiyne layer, in accordance with some embodiments.

FIG. 9 schematically depicts a plurality of electrically conductive pads and associated electrical paths utilized in the sensor of FIG. 6 for measuring electrical resistance of the graphene nano-flakes, in accordance with some embodiments.

FIG. 10 schematically depicts an embodiment of a sensor according to the present teachings, which includes an analysis sensing unit and a calibration sensing unit, in accordance with some embodiments.

FIG. 11 schematically depicts an embodiment of a sensor according to the present teachings, which includes a plurality of sensing units, in accordance with some embodiments.

FIGS. 12A and 12B depict an embodiment of a sensor according to the present teachings, which includes a microfluidic device for delivering a sample onto a sensing unit that comprises antibody- and/or aptamer-functionalized graphene nano-flakes, in accordance with some embodiments.

FIG. 13 schematically depicts a sensor according to an embodiment, which includes a reference electrode to which an AC voltage can be applied, in accordance with some embodiments.

In accordance with common practice, the various features illustrated in the drawings may not be drawn to scale. Accordingly, the dimensions of the various features may be arbitrarily expanded or reduced for clarity. In addition, some of the drawings may not depict all of the components of a given system, method or device. Finally, like reference numerals may be used to denote like features throughout the specification and figures.

### DETAILED DESCRIPTION

The present teachings are generally directed to sensors that employ antibody- and/or aptamer functionalized allotropes of carbon (e.g., graphene, graphene flakes and/or graphdiyne) for detecting prostate-specific biomarkers in a sample. As discussed in more detail below, in many embodiments, one or more graphene layers can be deposited on an underlying substrate, e.g., in the form of a single layer or multiple stacked layers, and functionalized with an antibody and/or an aptamer that specifically binds with a prostate-specific biomarker. A sample under investigation can be introduced onto the antibody- and/or aptamer-functionalized layer. The interaction of the prostate-specific biomarker, if present in the sample, with the antibody- and/or aptamer-functionalized layer (e.g., a graphene, a graphene flakes and/or a graphdiyne layer) can mediate a change in at least one electrical property of the functionalized layer, e.g., electron mobility of that layer, which can manifest itself as a change in one or more electrical properties of functionalized layer's, e.g., the layer's DC electrical resistance.

An analyzer can detect such a change and analyze it to determine whether the prostate-specific biomarker is present in the sample. In some embodiments, calibration methods can be employed to quantify the prostate-specific biomarker present in the sample. In other embodiments, the sensor can include a plurality of graphene flakes and/or a layer of graphdiyne that is functionalized with one or more antibodies and/or aptamers for specific binding to a prostate-specific biomarker.

Various terms are used herein in accordance with their ordinary meanings in the art. For example, the term “graphene” refers to a form of elemental carbon that is composed of a single sheet of carbon atoms.

The terms “graphene nano-flake,” “graphene flake,” and “graphene nanodot” are used herein interchangeably and refer to a plurality of hexagonal sp²-hybridized carbon rings that are fused together. In some embodiments, a plurality of graphene nano-flakes can be distributed so as to form a single layer while in other embodiments, a plurality of graphene nano-flakes are distributed such that at least some of the graphene nano-flakes are stacked on one another.

The term “graphdiyne” refers to an allotrope of carbon that is composed of sp and sp² hybridized carbon atoms, which can be constructed by replacing some carbon-carbon bonds in graphene with uniformly distributed diacetylenic linkages.

The term “analyte” as used herein refers to any molecular species whose detection in a sample is desired. For example, an analyte can be a protein, such as an antigen, or a pathogen, such as a bacterium.

The term “antibody” as used herein refers to a polypeptide exhibiting specific binding affinity, e.g., an immunoglobulin chain or fragment thereof, comprising at least one functional immunoglobulin variable domain sequence. An antibody encompasses full length antibodies and antibody fragments. In some embodiments, an antibody comprises an antigen binding or functional fragment of a full length antibody, or a full length immunoglobulin chain. For example, a full-length antibody is an immunoglobulin (Ig) molecule (e.g., an IgG antibody) that is naturally occurring or formed by normal immunoglobulin gene fragment recombinatorial processes. In embodiments, an antibody refers to an immunologically active, antigen-binding portion of an immunoglobulin molecule, such as an antibody fragment. An antibody fragment, e.g., functional fragment, comprises a portion of an antibody, e.g., Fab, Fab′, F(ab′)2, F(ab)2, variable fragment (Fv), domain antibody (dAb), or single chain variable fragment (scFv). A functional antibody fragment binds to the same antigen as that recognized by the intact (e.g., full-length) antibody.

The term “antibody” also encompasses whole or antigen binding fragments of domain, or single domain, antibodies, which can also be referred to as “sdAb” or “VHH.” Domain antibodies comprise either V_{H} or V_{L} that can act as stand-alone, antibody fragments. Additionally, domain antibodies include heavy-chain-only antibodies (HCAbs). Antibody molecules can be monospecific (e.g., monovalent or bivalent), bispecific (e.g., bivalent, trivalent, tetravalent, pentavalent, or hexavalent), trispecific (e.g., trivalent, tetravalent, pentavalent, hexavalent), or with higher orders of specificity (e.g., tetraspecific) and/or higher orders of valency beyond hexavalency. An antibody molecule can comprise a functional fragment of a light chain variable region and a functional fragment of a heavy chain variable region, or heavy and light chains may be fused together into a single polypeptide.

In some embodiments, an antibody is a glycoprotein produced by B lymphocytes in response to stimulation with an immunogen. An antibody can be composed of 4 polypeptides—2 heavy chains and 2 light chains—bound together by disulfide bonds to form a Y-shaped molecule.

The term “about” as used herein to qualify a numerical value is intended to denote a maximum variation of 10% about the numerical value.

The term “aptamer,” as used herein, refers to an oligonucleotide or a peptide molecule that exhibits specific binding to a target molecule. Aptamers are typically created by selecting them from a large random pool of oligonucleotide or peptide sequences, but natural aptamer do also exist.

Prostate-specific antigen (PSA) is a protein produced by cells of the prostate gland. PSA is also known as gamma-seminoprotein or kallikrein-3 (KLK3), and is a glycoprotein enzyme encoded in humans by the KLK3 gene. PSA is a member of the kallikrein-related peptidase family and is secreted by the epithelial cells of the prostate gland. PSA test methods according to the present teachings can measure the level of PSA (e.g., ng/mL) in the serum of a man. The blood level of PSA can be used as a potential indicator for prostate cancer. In addition to prostate cancer, a number of benign (not cancerous) conditions such as an enlarged or inflamed prostate can also cause a man's PSA level to increase. In general, however, it is considered that a man with higher PSA level is more likely to have prostate cancer. Further, a gradual increase of the PSA level over time in a same person may be a sign of prostate cancer. The PSA test is also used to monitor patients with a history of prostate cancer for recurrence of the disease. In addition or alternative to the PSA, other biomarkers such as kallikrein-related peptidase 2, prostate cancer antigen 3 (PCA3), TMPRSS2-ERG gene fusion, and the like can be used as potential indicators for prostate disease.

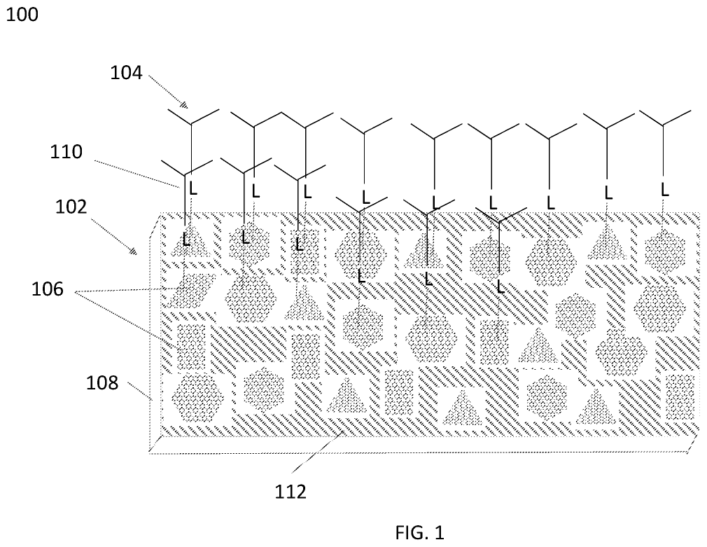

FIG. 1 schematically depicts an embodiment of a sensor 100 according to the present teachings that can be used for detecting an analyte, and more specifically a prostate-specific biomarker, in a sample. The sensor 100 includes a sensing element 102 having a graphene layer 106 that is deposited on an underlying substrate 108, where the graphene layer is functionalized with a plurality of antibodies and/or aptamers 104.

Alternatively or additionally, the sensor 100 according to the present teachings can include a graphene flake and/or graphdiyne-based sensing element. In some embodiments, such a sensing element can include a plurality of graphene flakes, a graphdiyne layer and/or a plurality of graphdiyne flakes, which are deposited on an underlying substrate 108. The sensing element can be functionalized with a plurality of antibodies and/or aptamers. In all embodiments disclosed in the present teachings, the graphene flakes can be partly or entirely substituted with graphdiyne flakes.

With continued reference to FIG. 1, in this embodiment, the graphene layer 106 is functionalized with the antibodies and/or aptamers 104 that exhibit specific binding to a prostate-specific biomarker. In this embodiment, a linker 110 is employed to couple the antibodies and/or aptamers 104 to the graphene layer 106. The linker can be coupled at one end thereof to the graphene layer 106 via π-π interaction and can be attached to the antibody and/or aptamer via a covalent bond at another end thereof.

A variety of linkers can be employed in the practice of the present teachings. By way of example, in some embodiments, the linker can be 1-pyrenebutonic acid succinimidyl ester. It has been discovered that this linker can be used to attach a variety of different antibodies and/or aptamers to a graphene layer, and hence it is expected that it can be similarly employed to attach a variety of different antibodies and/or aptamers to graphene flakes and/or a graphdiyne layer.

In some embodiments, the sensor 100 includes a passivation layer 112 that is disposed on the substrate to cover the areas of the substrate surface that are free of graphene (or graphene flakes and/or graphdiyne) and/or cover the portions of the graphene layer (or portions of the graphene flakes and/or the graphdiyne layer) that are not functionalized. In some embodiments, the passivation layer can be formed using Tween 20, BLOTTO, BSA (Bovine Serum Albumin) and/or gelatin, amino-PEGS-alcohol (pH 7.4). Further details regarding suitable linkers and passivating agents can be found, e.g., in U.S. Pat. No. 9,664,674, which is herein incorporated by reference in its entirety.

As noted above, the graphene layer 106 (or graphene flakes and/or graphdiyne layer) can be functionalized with a plurality of antibodies and/or aptamers 104 that exhibit specific binding to a prostate-specific biomarker. By way of example, the graphene layer 106 (or graphene flakes and/or graphdiyne layer) can be functionalized with an antibody and/or aptamer 104 that exhibits specific binding to a prostate-specific biomarker, such as prostate-specific antigen (PSA). By way of example, in some embodiments, the antibody can be anti-human PSA monoclonal antibody marketed by American Research Products, Inc. under Cat #03-10815 or Boster Bio under Cat #M01505; recombinant PSA antibody marketed by NSJ Bioreagents under Cat #V7293; or anti-KLK3 antibody marketed by St. John's Laboratory under Cat #STJ24332.

In addition, or instead, the graphene layer 106 (or graphene flakes and/or graphdiyne layer) can be functionalized with a plurality of antibodies and/or aptamers 104 that exhibit specific binding to other prostate-specific biomarkers. By way of example, the graphene layer 106 (or graphene flakes and/or graphdiyne layer) can be functionalized with an antibody and/or an aptamer 104 that exhibits specific binding to kallikrein-related peptidase 2 (KLK2), prostate cancer antigen 3 (PCA3), TMPRSS2-ERG gene fusion, or the like. By way of example, in some embodiments, mouse monoclonal anti-KLK2 antibodies such as Cat #ab40749 by Abcam and Cat #TAB-0622CL by Creative Bio Labs; an anti-PCA3 antibody under a tradename Progensa® PCA3 from Gen-Probe; and a recombinant anti-TMPRSS2 antibody marketed by Abcam under Cat #ab242384) can be employed as the antibodies 104.

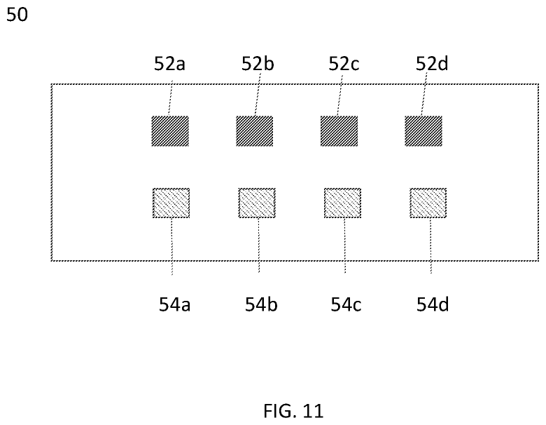

Further, in some embodiments, a sensor according to the present teachings can include one sensing unit that is configured to detect a prostate-specific biomarker and one or more other sensors that are configured to detect other biomarkers of interest. By way of example, with reference to FIG. 11, such a sensor 50 can include a sensing unit 52a that is configured to detect a prostate-specific biomarker and another sensing unit 54a that is configured to detect another biomarker of interest. In some embodiments, the biomarkers of interest can be prostate-specific biomarkers described above, and in other embodiments, the biomarkers of interest can be the biomarkers specific to other type of cancers, other diseases, or other health conditions.

By way of example, one or more of sensing units 52a, 52b, 52c, and 52d can include a graphene layer (or graphene flakes and/or graphdiyne layer) that is functionalized with an antibody and/or an aptamer that exhibits specific binding to a prostate-specific biomarker, and one or more of sensing units 54a, 54b, 54c, and 54d can include a graphene layer (or graphene flakes and/or graphdiyne layer) that is functionalized with an antibody and/or an aptamer that exhibits specific binding to another type of biomarker. For example, the sensing units 54a, 54b, 54c, and/or 54d can be functionalized with an antibody and/or an aptamer that exhibits specific binding to any of troponin, e.g., a particular isoform of troponin, C-reactive protein, B-type natriuretic peptide, or myeloperoxidase. In some embodiments, the anti-biomarker antibodies are monoclonal antibodies that exhibit specific binding to a particular isoform of the biomarker, e.g., a specific isoform of troponin. In other embodiments, the anti-biomarker antibodies can be polyclonal antibodies that exhibit binding to multiple isoforms of the biomarker. By way of example, in some embodiments, the graphene layer (or graphene flakes and/or graphdiyne layer) can be functionalized with cardiac troponin T (cTnT) and/or cardiac troponin I (cTnI).

In some embodiments, the graphene layer (or graphene flakes and/or graphdiyne layer) of the sensing units 54a, 54b, 54c, and/or 54d can be functionalized with an antibody and/or an aptamer 104 that exhibits specific binding to hemoglobin protein, e.g., human hemoglobin protein. By way of example, an anti-human hemoglobin antibody can be obtained from Sigma Aldrich under the product code H4890-2ML.

In some embodiments, the graphene layer 106 (or graphene flakes and/or graphdiyne layer) can be functionalized with protein G (PrG), which can be coupled to the underlying graphene layer (or graphene flakes and/or graphdiyne layer) via π-π interaction. The antibodies and/or aptamers 104 can be then covalently attached to the PrG. In some embodiments, the PrG can advantageously orient the antibodies and/or aptamers 104 so as to enhance the detection of a target analyte (e.g., a biomarker, such as a prostate-specific biomarker).

In some embodiments, the graphene layer (or graphene flakes and/or graphdiyne layer) deposited on an underlying substrate (e.g., plastic, or semiconductor such as silicon) can be incubated with the linker molecule (e.g., a 5 mM solution of 1 pyrenebutonic acid succinimidyl ester) for a few hours (e.g., 2 hours) at room temperature. The linker modified graphene layer (or graphene flakes and/or graphdiyne layer) can then be incubated with an antibody (e.g., 14D5) in a buffer solution (e.g., NaCO3—NaHCO3 (pH 9) at a selected temperature and for a selected duration (e.g., 7-10 hours at 4° C.), followed by rinsing with deionized (DI) water and phosphate buffered solution (PBS). In some cases, in order to quench the unreacted succinimidyl ester groups, the antibody- and/or aptamer-functionalized graphene layer (or graphene flakes and/or graphdiyne layer) can be incubated with ethanolamine (e.g., 0.1 M solution at a pH of 9 for 1 hour).

Without being bound to any particular theory, the interaction between a prostate-specific biomarker (or other biomarkers) and the antibody- and/or aptamer-functionalized graphene layer 106 (or graphene flakes and/or graphdiyne layer) can result in a change in at least one electrical property of the underlying graphene layer (or graphene flakes and/or graphdiyne layer), such as electron mobility that can in turn manifest itself as a change in the electrical resistance of the underlying layer of graphene (or graphene flakes and/or graphdiyne layer).

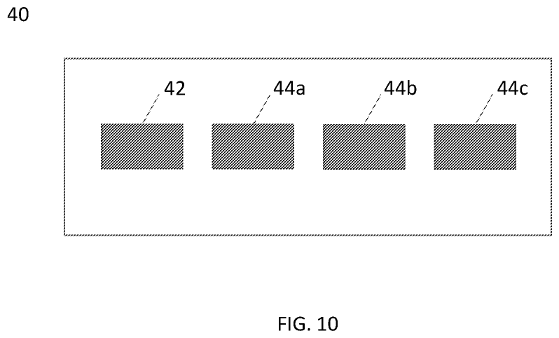

A plurality of electrode pads, such as those depicted in FIG. 9 can be coupled, via electrically conductive paths, to the antibody- and/or aptamer-functionalized graphene layer (or graphene flakes and/or graphdiyne layer) to allow the measurement of an electrical resistance thereof.

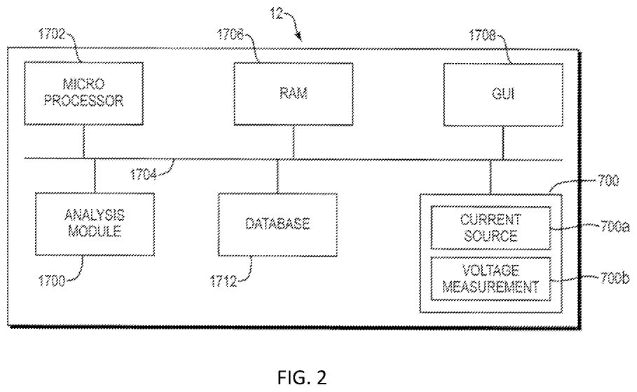

As shown in FIG. 2, an analyzer 12 can measure such a change in the electrical resistance (and/or other electrical property) of the graphene layer (or graphene flakes and/or graphdiyne layer) and determine whether the measured change correlates with the presence of a prostate-specific biomarker (and/or other biomarkers, such as those described above) in the sample. For example, in some embodiments, when the detected change in the electrical property of the graphene layer (or graphene flakes and/or graphdiyne layer) exceeds a certain threshold, the analyzer can indicate the presence of the target biomarker (e.g., a prostate-specific biomarker) in the sample under investigation.

In this embodiment, the analyzer 12 includes a data acquisition unit (herein also referred to as a measurement unit) 700, and an analysis module 1700. The analyzer can also include other components, such as a microprocessor 1702, a bus 1704, a Random Access Memory (RAM) 1706, a Graphical User Interface (GUI) 1708 and a database storage device 1712. The bus 1704 can allow communication among the different components of the analyzer. In some embodiments, the analysis module can be implemented in the form of a plurality of instructions stored in the RAM 1706. In other embodiments, it can be implemented as a dedicated hardware for performing processing of data obtained by the data acquisition unit 700.

Data acquisition unit 700 may be configured to acquire electrical data from which one or more electrical properties of the sensor (e.g., its DC resistance) can be determined. In this embodiment, the data acquisition unit 700 includes a current source 700a for supplying electrical currents of selected values to the sensing elements (e.g., to the graphene layer of the sensing elements) and a voltage measuring circuit 700b that can measure the voltage across each of the sensing elements, e.g., across the graphene layer (or graphene flakes and/or graphdiyne layer) of each sensing element. In other embodiments, a voltage source can be employed to apply a fixed voltage across the functionalized graphene layer (or graphene flakes and/or graphdiyne layer) and a change in the current following through the graphene layer (or graphene flakes and/or graphdiyne layer) in response to the interaction of a sample with the functionalized graphene layer (or graphene flakes and/or graphdiyne layer) can be measured to determine whether a biomarker of interest is present in the sample.

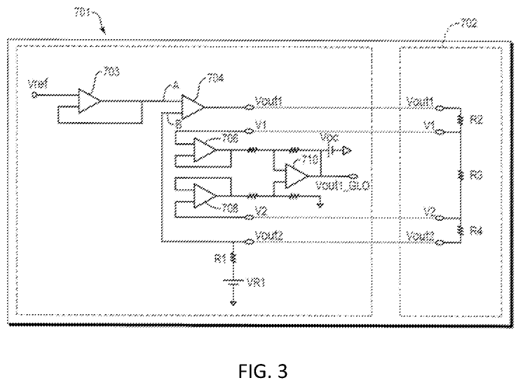

FIG. 3 schematically depicts a voltage measurement circuitry 701 according to some embodiments. Voltage measurement circuitry 701 can be employed as the measurement unit 700 for measuring electrical resistance of a sensor, e.g., sensor 702 that is depicted in this figure as an equivalent circuit diagram of a sensor according to the present teachings. A fixed voltage V (e.g., 1.2 V) is generated at the output of a buffer operational amplifier 703. This voltage is applied to one input (A) of a downstream operational amplifier 704 whose other input B is coupled to VR1 ground via a resister R1 The output of the operational amplifier 704 (Vout1) is coupled to one end of the sensor 702 and the non-connected to VR1 end of the resistor R1 is coupled to the other end of the sensor 702 (in this schematic diagram, resistor R2 denotes the resistance between two electrode pads at one end of a sensor, resistor R3 denotes the resistance of the sensor extending between two inner electrode pads of the sensor, and resistor R4 denotes the resistance between two electrode pads at the other end of the sensor). As the operational amplifier maintains the voltage at the non-connected to VR1 end of the resistor R1 at the fixed voltage applied to its input (A), e.g., 1.2 V, a constant current source is generated that provides a constant current flow through the sensor 702 and returns to ground via the resistor R1 and VR1.

In this embodiment, a voltage generated across the sensor can be measured via the two inner electrodes of the sensor. Specifically, one pair of the inner electrode pads is coupled to a buffer operational amplifier 706 and the other pair is coupled to the other buffer operational amplifier 708. The outputs of the buffer operational amplifiers are applied to the input ports of a differential amplifier 710 whose output port provides the voltage difference across the sensor. This voltage difference (Vout1_GLO) can then be used to measure the resistance exhibited by the sensor. The current forced through R3 is set by I=(Vref−VR1)/R1. The value of VR1 is digitally controlled. For each ⁻value. of current I, the corresponding voltage (Vout1_GLO) is measured and stored. The resistance of the sensor may be different at any given current so it is calculated as derivative of voltage, Vout1_GLO, with respect to current I, i.e., R=R=dV/dI≈ΔV/ΔI using the stored voltage versus current I. If the sensor has linear constant resistance, the value of R can be found as R=dV/dI=ΔV/ΔI=V/I.

Referring back to FIG. 2, the analysis module 1700 can be configured to receive the current and voltage values generated and obtained by the measurement unit 700 and can process these values according to the present teachings. The analysis may identify and quantify selected species, e.g., molecular species, present in a sample. Different units in the analyzer 12, as well as other units of the analysis module, can operate under the control of the microprocessor 1702.

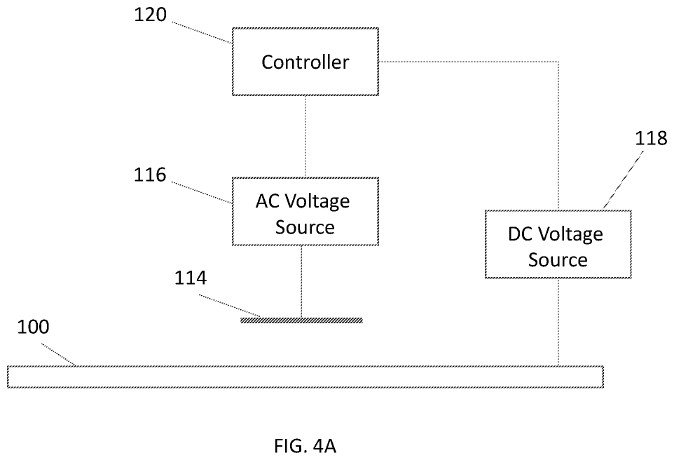

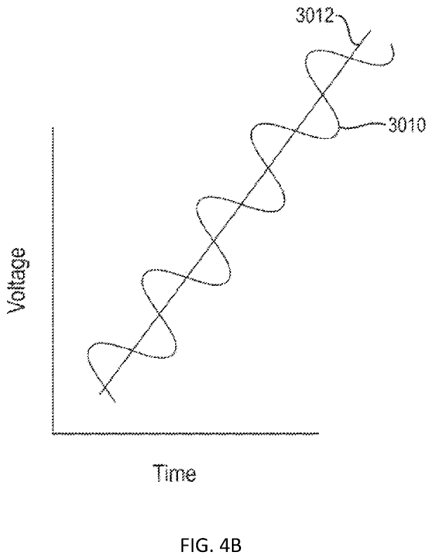

Referring to FIG. 4A, in some embodiments, the sensor 100 can include a reference electrode 114 disposed in proximity of the antibody- and/or aptamer-functionalized graphene layer (or graphene flakes and/or graphdiyne layer) (not shown in this figure for the sake of clarity), e.g., at a distance in a range of about 50 micrometers to about a few millimeters (e.g., 1-2 millimeters), e.g., on the side or above the functionalized graphene layer (or graphene flakes and/or graphdiyne layer). In some embodiments, the distance of the reference electrode 114 relative to the antibody- and/or aptamer-functionalized graphene layer (or graphene flakes and/or graphdiyne layer) can be in a range of about 100 microns to about 1 millimeter, or in a range of about 200 microns to about 0.5 millimeter. Further, in some embodiments, rather than being positioned above the graphene layer (or graphene nanoflakes and/or graphdiyne layer), the reference electrode 114 can be positioned in the same plane as the graphene layer (or graphene flakes and/or graphdiyne layer).

The reference electrode 114 can be utilized to generate a time-varying electric field at the interface of the antibody- and/or aptamer-functionalized graphene layer (or graphene nano-flakes and/or graphdiyne layer) and a liquid sample, e.g., a liquid sample suspected and/or expected of containing an analyte (e.g., a prostate-specific biomarker), that is brought into contact with that layer. For example, in this embodiment, an AC voltage source 116 can be programmed to apply an AC voltage to the reference electrode 114, which can in turn result in the generation of a time-varying electric field in the space between the reference electrode 114 and the functionalized graphene layer (or graphene flakes and/or graphdiyne layer).

The AC reference electrode 114 can be formed of any suitable electrical conductor. Some examples of suitable conductors include, without limitation, silver, copper, and gold. In some embodiments, the thickness of the reference electrode 114 can be, for example, in a range of about 100 nm to about 400 micrometers (microns), e.g., in a range of about 1 microns to about 100 microns, though other thicknesses can also be employed.

The application of such a time-varying electric field via the reference electrode 114 to the interface between the graphene layer (or graphene flakes and/or graphdiyne layer) and a liquid sample in contact with the graphene layer (or graphene flakes and/or graphdiyne layer) can advantageously facilitate the detection of one or more electrical properties of the antibody- and/or aptamer-functionalized graphene layer (or graphene flakes and/or graphdiyne layer), e.g., a change in its resistance in response to its interaction with an analyte of interest (e.g., a prostate-specific biomarker) present in the sample that exhibits specific binding to the antibody and/or aptamer of the functionalized graphene layer (or graphene flakes and/or graphdiyne layer). In particular, it has been discovered that the application of an AC voltage having a frequency in a range of about 1 kHz to about 1 MHz, e.g., in a range of about 10 kHz to about 500 kHz, or in a range of about 20 kHz to about 400 kHz, or in a range of about 30 kHz to about 300 kHz, or in a range of about 40 kHz to about 200 kHz, can be especially advantageous in this regard. By way of example, the amplitude of the AC voltage applied to the reference electrode 114 can be in a range of about 1 millivolt to about 3 volts, e.g., in a range of about 100 millivolts to about 2 volts, or in range of about 200 millivolts to about 1 volt, or in range of about 300 millivolts to about 1 volt, e.g., in a range of about 0.5 volts to 1 volt.

Further, in some cases, the voltage applied to the reference electrode 114 can have an AC component and a DC offset, where the DC offset can be in a range of about −40 volts to about +40 volts, e.g., −1 volt to about +1 volt. More specifically, a controller 120 is programed to control an AC voltage source 116 and a DC voltage source 118. Although in this embodiment the AC voltage source 116 and the DC voltage source 118 are shown as two independent units, in other embodiments the functionalities of the AC voltage source 116 for applying an AC voltage and a DC offset voltage to the reference electrode 114 and the functionalities of a power supply can be combined in a single unit.

The controller 120 can be implemented in hardware, software, and/or firmware in a manner known in the art as informed by the present teachings. For example, the controller 120 can have the components illustrated in FIG. 3 for the analyzer.

By way of illustration, FIG. 4B schematically depicts a combination of an AC voltage 3010 and a DC offset voltage 3012 applied to the reference electrode 3001. By way of example, the DC offset voltage can extend from about −10 V to about 10 V (e.g., from −1 V to about 1 V), and the applied AC voltage can have the frequencies and amplitudes disclosed above.

Without being limited to any particular theory, in some embodiments, it is expected that the application of such a voltage to the reference electrode 114 can minimize, and preferably eliminate, an effective capacitance associated with a sample, e.g., a liquid sample, with which the functionalized graphene layer (or graphene flakes and/or graphdiyne layer) are brought into contact as the sample is being tested, thereby facilitating the detection of a change in the resistance of the underlying graphene layer (or graphene flakes and/or graphdiyne layer) in response to the interaction of the antibodies and/or aptamers with a respective analyte. In some cases, the effective capacitance of the sample can be due to ions present in the sample.

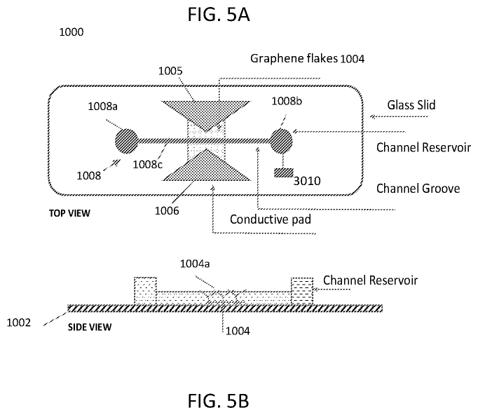

By way of example, FIGS. 5A and 5B schematically depict an example of a device 1000 according to an embodiment of the present teachings for detecting a prostate-specific biomarker in a sample. The device 1000 includes a substrate 1002 on a top surface of which a graphene layer 1004 (or graphene flakes and/or graphdiyne layer) is deposited.

A variety of different substrates can be employed. By way of example, the substrate 1002 can be any of a semiconductor, such as silicon, or glass or plastic. In some embodiments in which the substrate 1002 is formed of silicon, a layer of silicon oxide can separate the upper layer of graphene (or graphene flakes and/or graphdiyne layer) from the underlying silicon layer.

Similar to the previous embodiment, in some embodiments, the antibodies and/or aptamers 1004a are coupled to the underlying graphene layer (or graphene flakes and/or graphdiyne layer) via a linker, where the linker is attached via π-π at one end thereof to the graphene layer 1004 (or graphene flakes and/or graphdiyne layer). The antibodies and/or aptamers 1004a can be attached to the other end of the linker, e.g., via a covalent bond. By way of example, in some embodiments, 1-pyrenebutonic acid succinimidyl ester can be employed as the linker to facilitate the coupling of the antibody molecules to the underlying graphene layer (or graphene flakes and/or graphdiyne layer). It has been discovered that 1-pyrenebutonic acid succininmidyl ester can be used to couple a variety of different antibodies to an underlying graphene layer (or graphene flakes and/or graphdiyne layer). As such, it is expected that this linker can be used for coupling antibodies and/or aptamers that specifically bind to a prostate-specific biomarker to an underlying layer of graphene (or graphene flakes and/or graphdiyne layer), where the antigen-antibody interaction or antigen-aptamer interaction can mediate a change in one or more electrical properties of the underlying layer of graphene (or graphene flakes and/or graphdiyne layer).

In some embodiments, the graphene layer (or graphene flakes and/or graphdiyne layer) can be incubated with the linker molecule (e.g., a 5 mM solution of 1-pyrenebutonic acid succimidyl ester) for a few hours (e.g., 2 hours) at room temperature to ensure coupling of the linker molecules to the underlying graphene layer (or graphene flakes and/or graphdiyne layer). The linker modified graphene layer (or graphene flakes and/or graphdiyne layer) can then be incubated with an antibody and/or an aptamer of interest in a buffer solution (e.g., NaCO3—NaHCO3 buffer solution (pH 9)) at a selected temperature and for a selected duration (e.g., 7-10 hours at 4° C.), followed by rinsing with deionized (DI) water and phosphate buffered solution (PBS). In order to quench the unreacted succinimidyl ester groups, the modified graphene layer (or graphene flakes and/or graphdiyne layer) can be incubated with ethanolamine (e.g., 0.1 M solution at a pH of 9 for 1 hour).

Subsequently, the non-functionalized areas of the graphene layer (or graphene flakes and/or graphdiyne layer) and/or the substrate can be passivated via a passivation layer. By way of example, the passivation of the non-functionalized portions of the graphene layer (or graphene flakes and/or graphdiyne layer) and/or the substrate can be achieved, e.g., via incubation with 0.1% Tween 20 or BLOTTO, BSA (Bovine Serum Albumin), and gelatin and/or amino-PEGS-alcohol (pH 7.4). Further details regarding the use of linkers suitable for use in the practice of the invention can be found, e.g., in U.S. Pat. No. 9,664,674, which is herein incorporated by reference in its entirety.

Referring again to FIGS. 5A and 5B, two metallic conductive pads 1005/1006 in electrical contact with the graphene layer 1004 (or graphene flakes and/or graphdiyne layer) allow measuring the electrical resistance of the graphene layer 1004 (or graphene flakes and/or graphdiyne layer), and particularly, a change in the electrical resistance of the graphene layer 1004 (or graphene flakes and/or graphdiyne layer) in response to exposure thereof to a sample containing a prostate-specific biomarker. In some embodiments, the electrically conductive pads 1005/1006 can be formed of silver high conductive paste, though other electrically conductive materials can also be employed. The conductive pads 1005/1006 can be electrically connected to a measurement device, e.g., a voltmeter, via a plurality of conductive wires for measuring the Ohmic electrical resistance of the graphene layer (or graphene flakes and/or graphdiyne layer).

The device 1000 further includes a microfluidic structure 1008 having two reservoirs 1008a/1008b and a fluid channel 1008c that fluidly connects the two reservoirs 1008a/1008b. As shown more clearly in FIG. 5B, the fluid channel 1008c can be arranged such that a portion thereof is in fluid contact with a portion of the graphene layer 1004 (or graphene flakes and/or graphdiyne layer).

In some embodiments, in use, a sample containing a prostate-specific biomarker can be introduced into one of the reservoirs 1008a/1008b and can be made to flow, e.g., via application of hydrodynamic pressure thereto, to the other reservoir through the microfluidic channel 1008c. In this embodiment, a pump 3010 can be coupled to a reservoir 1008b to facilitate the flow of the sample to the other reservoir 1008a. In other embodiments, the pump 3010 may be coupled to the other reservoir 1008a and/or to a fluid channel 1008c connecting those reservoirs 1008a/1008b.

The passage of the sample through the channel 1008c brings the biomarker (e.g., a prostate-specific biomarker), if any, present in the sample into contact with the antibody- and/or aptamer-functionalized graphene layer 1004 (or graphene flakes and/or graphdiyne layer). Without being limited to any particular theory, the interaction of the biomarker with the antibodies and/or aptamers to which they can bind can mediate a change in the electrical conductivity (and hence resistance) of the underlying graphene layer 1004 (or graphene flakes and/or graphdiyne layer), e.g., via charge transfer or other mechanisms. This change in the electrical conductivity of the graphene layer 1004 (or graphene flakes and/or graphdiyne layer) can in turn be measured to detect the presence and quantity of the biomarker in the sample under study.

In some embodiments, a four-point measurement technique can be used to measure the resistance of the antibody- and/or aptamer-functionalized graphene layer (or graphene flakes and/or graphdiyne layer) in response to exposure thereof to a sample under investigation.

A voltage measuring device, such as the above voltage measuring circuitry 701, can be employed to measure a change in the electrical resistance of the underlying graphene layer (or graphene flakes and/or graphdiyne layer) in response to the interaction of a biomarker with the antibodies and/or aptamers coupled to the graphene layer (or graphene flakes and/or graphdiyne layer).

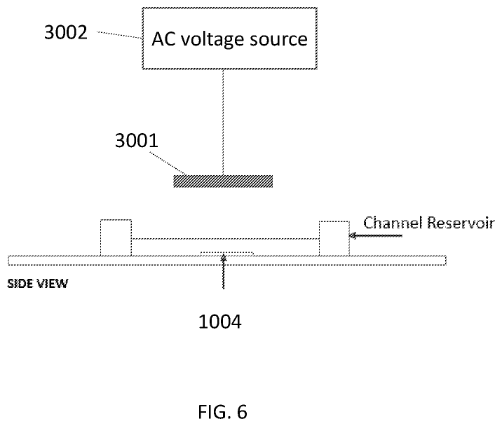

Referring to FIG. 6, in some embodiments, the sensor 1000 according to an embodiment can include a reference electrode 3001 disposed in proximity of the antibody- and/or aptamer-functionalized graphene layer 1004 (or graphene flakes and/or graphdiyne layer), e.g., at a distance in a range of about 50 micrometers to about a few millimeters (e.g., 1-2 millimeters) above the antibody- and/or aptamer-functionalized graphene layer 1004 (or graphene flakes and/or graphdiyne layer). In some embodiments, the distance of the reference electrode 3001 relative to the functionalized graphene layer 1004 (or graphene flakes and/or graphdiyne layer) can be in a range of about 100 microns to about 1 millimeter, or in a range of about 200 microns to about 0.5 millimeter. Further, in some embodiments, rather than being positioned above the graphene layer 1004 (or graphene flakes and/or graphdiyne layer), the reference electrode 3001 can be positioned in the same plane as the graphene layer 1004 (or graphene flakes and/or graphdiyne layer).

The reference electrode 3001 can be utilized to generate a time-varying electric field at the interface of the functionalized graphene layer (or graphene flakes and/or graphdiyne layer) and a liquid sample, e.g., a liquid sample containing biomarkers (e.g., PSA), that is brought into contact with that layer. For example, in this embodiment, an AC voltage source 3002 can be programed to apply an AC voltage to the reference electrode, which can in turn result in the generation of a time-varying electric field in the space between the reference electrode and the functionalized graphene layer (or graphene flakes and/or graphdiyne layer).

The AC reference electrode 3001 can be formed of any suitable electrical conductor. Some examples of suitable conductors include, without limitation, silver, copper, and gold. In some embodiments, the thickness of the reference electrode 3001 can be, for example, in a range of about 100 nm to about 400 micrometers (microns), e.g., in a range of about 1 micron to about 100 microns, though other thicknesses can also be employed.

As discussed above, the application of such a time-varying electric field via the reference electrode 3001 to the interface between the graphene layer (or graphene flakes and/or graphdiyne layer) and a liquid sample in contact with the graphene layer (or graphene flakes and/or graphdiyne layer) can advantageously facilitate the detection of one or more electrical properties of the antibody- and/or aptamer-functionalized graphene layer (or graphene flakes and/or graphdiyne layer), e.g., a change in its resistance in response to its interaction with a biomarker (e.g., PSA) present in the sample that exhibits specific binding to the antibody of the functionalized graphene layer (or graphene flakes and/or graphdiyne layer). In particular, it has been discovered that the application of an AC voltage having a frequency in a range of about 1 kHz to about 1 MHz, e.g., in a range of about 10 kHz to about 500 kHz, or in a range of about 20 kHz to about 400 kHz, or in a range of about 30 kHz to about 300 kHz, or in a range of about 40 kHz to about 200 kHz, can be especially advantageous in this regard. By way of example, the amplitude of the AC voltage applied to the reference electrode can be in a range of about 1 millivolt to about 3 volts, e.g., in a range of about 100 millivolts to about 2 volts, or in range of about 200 millivolts to about 1 volt, or in range of about 300 millivolts to about 1 volt, e.g., in a range of about 0.5 volts to 1 volt. Further, in some cases, the voltage applied to the reference electrode can have an AC component and a DC offset, where the DC offset can be in a range of about −40 volts to about +40 volts, e.g., −1 volt to about +1 volt.

Without being limited to any particular theory, in some embodiments, it is expected that the application of such a voltage to the reference electrode can minimize, and preferably eliminate, an effective capacitance associated with a sample, e.g., a liquid sample, with which the functionalized graphene layer (or graphene flakes and/or graphdiyne layer) are brought into contact as the sample is being tested, thereby facilitating the detection of a change in the resistance of the underlying graphene layer (or graphene flakes and/or graphdiyne layer) in response to the interaction of the antibodies with a respective target analyte (e.g., PSA). In some cases, the effective capacitance of the sample can be due to ions present in the sample.

The present teachings can be applied to detect a variety of target analytes (e.g., PSA), such as those discussed above, in a variety of different samples. Some examples of samples that can be interrogated include, without limitation, bodily fluids, such as blood, urine, semen, saliva, etc. In some embodiments, the bodily fluids can be diluted or concentrated to adjust the concentration of the analytes for suitable detection range.

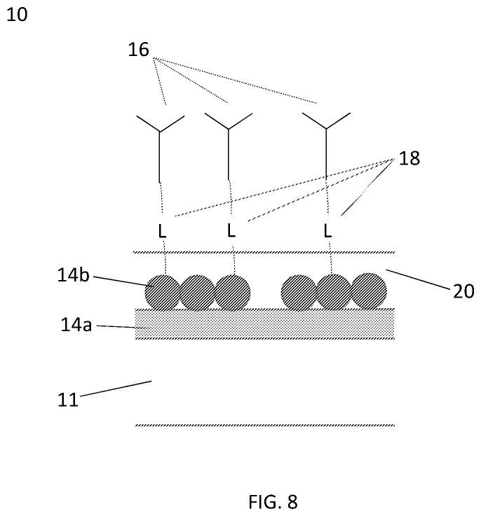

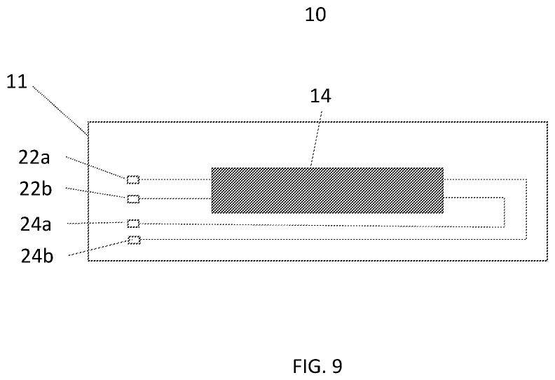

FIG. 8 schematically depicts a sensor 10 according to another embodiment of the present teachings. The sensor 10 comprises a multi-layer structure in which a plurality of graphene and/or graphdiyne flakes 14b are deposited on an underlying graphene or graphdiyne layer 14a. Any combination of the flakes 14b and the layer 14a are possible. In other words, a plurality of graphene flakes can be deposited on a graphene layer; a plurality of graphdiyne flakes can be deposited on a graphene layer; a plurality of graphene flakes and a plurality of graphdiyne flakes can be deposited on a graphene layer; a plurality of graphene flakes can be deposited on a graphdiyne layer; a plurality of graphdiyne flakes can be deposited on a graphdiyne layer; a plurality of graphene flakes and a plurality of graphdiyne flakes can be deposited on a graphdiyne layer.

In this embodiment, the graphene or graphdiyne flakes 14b (and/or graphene or graphdiyne layer 14a) are functionalized with a plurality of antibodies and/or aptamers 16. In other words, in this embodiment, the graphene or graphdiyne flakes 14b (and/or graphene or graphdiyne layer 14a) are functionalized with a plurality’ of antibodies and/or aptamers 16 that exhibit specific binding to a prostate-specific biomarker. By way of example, the prostate-specific ⁻biomarkers can include a prostate-specific antigen (PSA), kallikrein-related peptidase 2, prostate cancer antigen 3 (PCA3), TMPRSS2-ERG gene fusion, or the like.

Similar to the previous embodiments, a variety of linker molecules 18 can be employed for coupling the antibodies and/or aptamers 16 to the underlying graphene or graphdiyne flakes 14b (and/or graphene or graphdiyne layer 14a). By way of example, in some embodiments, 1-pyrenebutonic acid succinimidyl ester is employed as a linker to facilitate the coupling of the antibodies and/or aptamers 16 to the underlying graphene or graphdiyne flakes 14b (and/or graphene or graphdiyne layer 14a).

In this embodiment, the plurality of antibodies and/or aptamers 16 can cover a fraction of, or the entire, surface of the graphene or graphdiyne flakes 14b (and/or graphene or graphdiyne layer 14a). In various embodiments, the fraction can be at least about 60%, at least about 70%, at least about 80%, or 100% of the graphene flakes. The remainder of the graphene or graphdiyne flakes 14b (and/or graphene or graphdiyne layer 14a), i.e., the graphene flakes not functionalized and/or parts of the substrate that are free of graphene or graphdiyne flakes (and/or graphene or graphdiyne layer) can be passivated via a passivation layer 20. By way of example, the passivation layer can be formed by using Tween 20, BLOTTO, BSA (Bovine Serum Albumin), gelatin and/or amino-PEGS-alcohol (pH 7.4). The passivation layer can inhibit, and preferably prevent, the interaction of a sample of interest introduced onto the antibody- and/or aptamer-functionalized graphene or graphdiyne, flakes 14b (and/or graphene or graphdiyne layer 14a) with those graphene or graphdiyne flakes (and/or graphene or graphdiyne layer) that are not functionalized and/or parts of the substrate that are free of graphene or graphdiyne flakes (and/or graphene or graphdiyne layer). This can in turn lower the noise in the electrical signals that will be generated as a result of the interaction of the analyte of interest with the antibody and/or aptamer molecules.

By way of example, in some embodiments, a plurality⁻ of graphene or graphdiyne, flakes 14b (and/or graphene or graphdiyne layer 14a) deposited on an underlying substrate (e.g., plastic, a semiconductor, such as silicon, or a metal substrate, such as a copper film) can be incubated with the linker molecule (e.g., a 5 mM solution of 1-pyrenebutonic acid succinimidyl ester) for a few hours (e.g., 2 hours) at room temperature.

The linker modified graphene or graphdiyne flakes 14b (and/or graphene or graphdiyne layer 14a) can then be incubated with the antibody and/or aptamer of interest (e.g., a prostate-specific biomarker) in a buffer solution (e.g., NaCO3—NaHCO3 buffer solution (pH 9)) at a selected temperature and for a selected duration (e.g., 7-10 hours at 4° C.), followed by rinsing with deionized (DI) water and phosphate buffered solution (PBS). In order to quench the unreacted succinimidyl ester groups, the modified graphene or graphdiyne flakes 14b (and/or graphene or graphdiyne layer 14a) can be incubated with ethanolamine (e.g., 0.1 M solution at a pH of 9 for 1 hour).

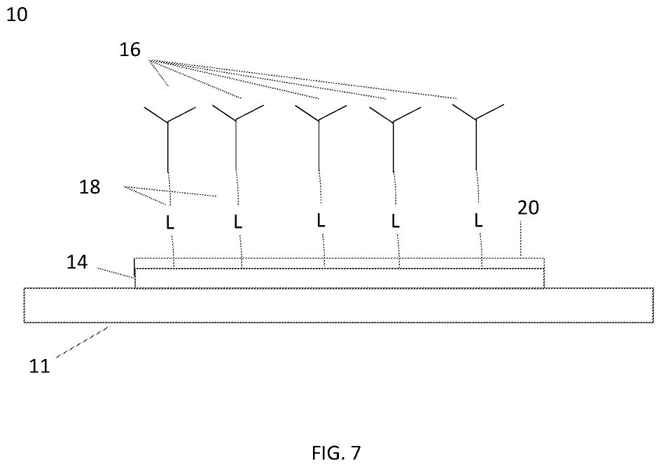

Similar to the previous embodiments, subsequently, the non-functionalized graphene or graphdiyne flakes (and/or non-functionalized areas of graphene or graphdiyne layer) can be passivated via a passivation layer, such as the passivation layer 20 schematically depicted in FIG. 7. By way of example, the passivation of the non-functionalized graphene or graphdiyne flakes (and/or non-functionalized areas of graphene or graphdiyne layer) can be achieved, e.g., via incubation with 0.1% Tween 20.

Similar to the previous embodiments and with reference to FIG. 9, the sensor 10 further includes electrically conductive pads 22a, 22b, 24a and 24b, that allow four point measurement of modulation of an electrical property of the functionalized graphene layer (or graphene flakes and/or graphdiyne layer) in response to interaction of antibodies and/or aptamers with the biomarkers coupled to the graphene layer 14 (or graphene flakes and/or graphdiyne layer). In particular, in this embodiment, the conductive pads 22a/22b are electrically coupled to one end of the functionalized graphene layer 14 (or graphene flakes and/or graphdiyne layer) and the conductive pads 24a/24b are electrically coupled to the opposed end of the functionalized graphene layer 14 for graphene flakes and/or graphdiyne layer) to allow measuring a change in an electrical property of the underlying graphene layer 14 (or graphene flakes and/or graphdiyne layer caused by the interaction of biomarkers (e.g., a prostate-specific biomarker) in a sample under study with the antibodies and/or aptamers that are coupled to the graphene layer 14 (or graphene flakes and/or graphdiyne layer).

By way of example, in this embodiment, a change in the DC resistance of the underlying graphene layer 14 (or graphene flakes and/or graphdiyne layer) can be monitored to determine the presence and/or concentration of biomarkers (e.g., a prostate-specific biomarker) in a sample under study in other embodiments, a change in electrical impedance of the graphene layer 14 (or graphene flakes and/or graphdiyne layer) characterized by a combination of DC resistance and capacitance of the grapheme/antibody (or graphene/aptamer) system can be monitored to determine whether the biomarkers (e.g., a prostate-specific biomarker) are present in a sample under study. The electrically conductive pads 22a, 22b, 24a, and 24b can be formed using a variety of metals, such as copper and copper alloys, among others.

An analyzer similar to that described above in connection with FIGS. 2 and 3 can be employed to measure a change in an electrical property of the antibody- and/or aptamer-functionalized graphene layer (or graphene flakes and/or graphdiyne layer). The analyzer can further include a module comprising instructions for analyzing the measured electrical signals generated by the functionalized grapheme layer (or graphene flakes and/or graphdiyne layer) to determine whether the biomarkers (e.g., a prostate-specific biomarker) are present in a sample, and optionally quantify the amount of the biomarkers in the sample, e.g., by comparing the measured electronic signal with a calibration signal. The analyzer can be implemented in hardware, firmware and/or software. By way of example, the analyzer can include a processor in communication, via one or more buses, with one or more memory modules including transient and permanent memory modules. The instructions for analyzing the data received from the sensor can be stored in at least one of the memory modules and the processor can operate on the data to analyze the signals.

In some embodiments, a sensor according to the present teachings can allow quantifying the amount of the ⁻biomarkers (e.g., a prostate-specific biomarker), if any, in a sample under study, e.g., a person's blood sample. By way of example, FIG. 10 schematically depicts such a sensor 40 that includes a test sensing element 42 and three calibration sensing elements 44a, 44b, and 44c. Each sensing element includes graphene layer (or graphene flakes and/or graphdiyne layer) functionalized with antibodies and/or aptamers and has a structure similar to that discussed above in connection with the sensor 10.

In an exemplary use, prior to testing a sample with PSA (or other prostate-specific biomarkers), calibration samples having different concentrations of PSA can be applied to the calibration sensing elements 44a, 44b, and 44c. Each calibration sample includes a different known amount of PSA. An electrical signal generated by the calibration sensor in response to contact with the calibration sample can be measured and used for quantifying the amount of PSA in a sample under study.

More specifically, each calibration sensing element 44a, 44b, and 44c can be used to obtain an electronic response of the functionalized graphene layer (or graphene flakes and/or graphdiyne layer) to PSA in one of the calibration samples. The responses of the three calibration sensing elements can be used to generate a calibration curve. While in this embodiment, three calibration sensing elements are employed, in other embodiments, the number of the calibration sensing elements can be more or less than three. A sample under study can be applied to the sample-testing sensing element 42, The calibration curve can be employed to quantify the amount of PSA in the test sample, if any, based on the measured change in the resistance of the underlying grapheme, layer (or graphene flakes and/or graphdiyne layer) in response to contact with the test sample.

In some embodiments, a sensor according to the present teachings can include an array of sensing elements that allow parallel measurements of different prostate-specific biomarkers or other biomarkers. By way of example, FIG. 11 schematically depicts such a sensor 50 having a plurality of sensing elements 52a, 52b, 52c, and 52d (herein collectively referred to as sensing elements 52) as well as sensing elements 54a, 54b, 54c, and 54d (herein collectively referred to as sensing elements 54). One or more of the sensing elements 52 and 54 includes a graphene layer (or graphene flakes and/or graphdiyne layer) that is functionalized with a prostate-specific biomarker and has a structure similar to that discussed above in connection with sensor 10. In this embodiment, one or more of the sensing elements 52 and 54 can be functionalized with other prostate-specific biomarkers or a different biomarker. By way of example, the prostate-specific biomarkers can include PSA, kallikrein-related peptidase 2, prostate cancer antigen 3 (PCA3), TMPRSS2-ERG gene fusion, or the like. By way of example, other biomarkers can include biomarkers specific to other type of cancers, other diseases, other health conditions, or the like.

In some embodiments, each of the sensing element 52 and 54 can have an associated calibration sensor, which can be employed to calibrate the corresponding sensing element in a manner discussed above.

An analyzer (not shown), similar to that described above, can be used to measure the change in the resistances of the sensing elements 52 and 54 in response to contact of those sensing elements with a sample under study. In some embodiments, the analyzer can employ a multiplexing circuitry to measure sequentially the resistance of each of the sensing elements.

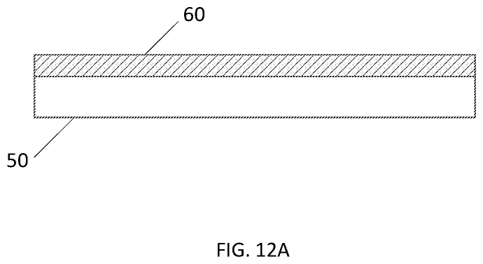

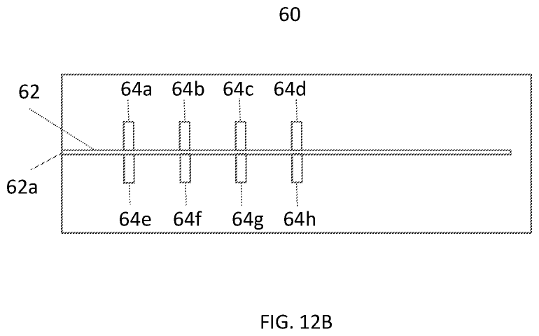

In some embodiments of the sensor 50, a fluidic deliver device can be employed to deliver a sample under study to the sensing elements 52 and 54. By way of example, FIGS. 12A and 12B schematically depict such a fluidic delivery device 60 that is fluidically coupled to the sensing elements 52 and 54. The fluid delivery device 60 includes a central capillary channel 62 having an input port 62a for receiving a sample and a plurality of peripheral capillary channels 64a, 64b, 64c, 64d, 64e, 64f, 64g, and 64h for delivering the sample to the sensing elements 52 and 54.

As discussed above, in some embodiments, each sensing element can have an associated calibration sensing element that allows calibrating the sensing element for quantifying the concentration of a biomarker in a sample under study. In this manner, a sensor having a plurality of sensing elements functionalized with a variety of different biomarkers can be used to not only identify the presence of that biomarker in a sample but also quantify its concentration.

A panel of a plurality of biomarkers can be used as a diagnostic tool for diagnosing one or more disease conditions. Further, in some cases, a panel of biomarkers can be used as a predictive tool.

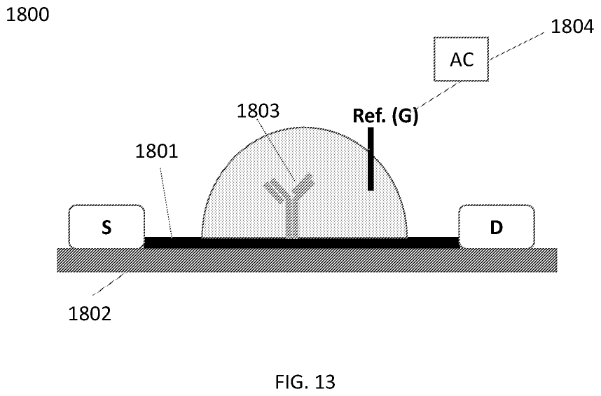

FIG. 13 schematically depicts another embodiment of a sensor 1800 according to the present teachings, which includes a graphene layer 1801 (or graphene flakes and/or graphdiyne layer) that is disposed on an underlying substrate 1802, e.g., a semiconductor substrate, and that is functionalized with an antibody and/or aptamer of interest 1803. A source electrode (S) and a drain electrode (D) are electrically coupled to the functionalized graphene layer 1801 (or graphene flakes and/or graphdiyne layer) to allow measuring a change in one or more electrical parameters of the functionalized graphene layer 1801 (or graphene flakes and/or graphdiyne layer) in response to interaction of the functionalized graphene layer 1801 (or graphene flakes and/or graphdiyne layer) with a sample. The sensor 1800 further includes a reference electrode (G) that is disposed in proximity of the graphene, layer (or graphene flakes and/or graphdiyne layer).

In use, in some embodiments, a change in the electrical resistance of the functionalized graphene layer 1801 (or graphene flakes and/or graphdiyne layer) can be measured in response to the interaction of the functionalized graphene layer 1801 (or graphene flakes and/or graphdiyne layer) with a sample to identify and optionally quantify an analyte of interest (e.g., a prostate-specific biomarker) in the sample. For example, when the sample includes an analyte that specifically binds to the antibody and/or aptamer 1803, the interaction of the antibody and/or aptamer 1803 with the analyte can modulate the electrical resistance of the graphene layer 1801 (or graphene flakes and/or graphdiyne layer). A measurement of such a modulation of the electrical resistance of the graphene layer 1801 (or graphene flakes and/or graphdiyne layer’ can be employed to identify that analyte (e.g., a prostate-specific biomarker) in a sample.

As noted above, it has been discovered that the application of an AC (alternating current) voltage via an AC voltage source 1804 to the graphene layer 1801 (or graphene flakes and/or graphdiyne layer) can facilitate the detection of one or more electrical properties of the functionalized graphene layer 1801 (for graphene flakes and/or graphdiyne layer), e.g., a change in its resistance in response to the interaction of the antibody and/or aptamer 1803 with an analyte exhibiting specific binding to the antibody and/or aptamer 1803. In particular, it has been discovered that the application of an AC voltage having a frequency in a range of about I kHz to 1 MHz, e.g., in a range of about 10 kHz to about 500 kHz or in a range of about 20 kHz to about 100 kHz, can be especially advantageous in this regard. By way of example, the amplitude of the AC voltage applied to the reference electrode can be in a range of about 1 millivolt to about 3 volts, e.g., 0.5 volts to 1 volt. Further, in some cases, the voltage applied to the reference electrode can have an AC component and a DC offset, where the DC offset can be in a range of about −40 volts to about +40 volts, e.g., −0.1 volt to about +1 volt.

Without being limited to any particular theory, in some embodiments, it is expected that the application of such a voltage to the reference electrode can minimize, and preferably eliminate, an effective capacitance associated with a sample, e.g., a liquid sample, with which the functionalized graphene layer 1801 (or graphene flakes and/or graphdiyne layer) is brought into contact as the sample is being tested, thereby facilitating the detection of a change in the resistance of the underlying graphene layer 1801 (or graphene flakes and/or graphdiyne layer) in response to the interaction of the antibodies and/or aptamer 1803 with a respective antigen. In some cases, the effective capacitance of the sample can be due to ions present in the sample.

A sensor according to the present teachings can be employed in a variety of settings. By way of example, a sensor according to the present teachings can be employed in a medical setting. Further, a sensor according to the present teachings can be employed for home use. In such cases, the analyzer can be implemented on a mobile device. In addition or alternatively, the analyzer can be implemented on a remote server that can be in communication with the sensor via a network, e.g., the Internet, to receive sensing data, such as a voltage measured across the antibody- and/or aptamer-functionalized graphene layer (or graphene flakes and/or graphdiyne layer). The analyzer can employ the sensing data to determine whether an analyte of interest is present in a sample under study in a manner discussed above.

The graphene flakes employed in various embodiments of the present teachings can be fabricated using a variety of different methods. Some such methods rely on bottom-up production of graphene flakes in which small molecular units are combined to form large aromatic hydrocarbons via a variety of chemical reactions. For example, the articles by J. Wu et al, published in Chem. Rev. 107 (2007) 718 and by Zhi et al. published in J. Mater. Chem. 18 (2008) 1472, which are herein incorporated by reference in their entirety, describe such bottom-up methods for fabricating graphene nano-flakes. These articles also describe adding a variety of terminations to these structures, including hydrogen and alkyl groups.

Other methods of generating graphene nano-flakes rely on top-down synthesis techniques. For example, in some such fabrication techniques, a piece of graphene (or graphene-related material such as graphene oxide) can be cut directly into graphene nano-flakes.

The graphdiyne layer and/or the graphdiyne flakes employed in various embodiments of the present teachings can be fabricated using a variety of different methods. Some such methods rely on on-surface synthesis (e.g., on Au, Ag, or Cu surfaces), top-down method, explosion method, and wet chemistry methods. For example, the articles by Gao et al. published in Chem. Soc. Rev. 38 (2019) 908-936 and by Jia et al. published in Acc. Chem. Res. 50 (2017) 343-349, which are herein incorporated by reference in their entirety, describe synthesis of graphdiyne.

In some embodiments, a mixture of graphene nano-flakes and/or graphdiyne flakes and a solvent (e.g., acetone can be spin-coated on a substrate, e.g., silicon or glass substrate, and then subsequently functionalized with a desired antibody and/or aptamer.

Those having ordinary skill in the art will appreciate that various changes can be made to the above embodiments without departing from the scope of the invention.

What is claimed is:

1. A sensor for detecting a prostate-specific biomarker, comprising:
a graphene layer deposited on an underlying substrate,
a plurality of antibodies coupled to the graphene layer to generate an antibody-functionalized graphene layer, wherein the antibodies exhibit specific binding to a prostate-specific biomarker, and
a plurality of electrical conductors electrically coupled to the antibody-functionalized graphene layer for measuring an electrical property thereof.

2. The sensor of claim 1, wherein the prostate-specific biomarker comprises a prostate-specific antigen (PSA).

3. The sensor of claim 1, further comprising a reference electrode disposed in proximity of the antibody-functionalized graphene layer.

4. The sensor of claim 3, further comprising an AC voltage source for applying an AC voltage to the reference electrode,

5. The sensor of claim 4, wherein the AC voltage has a frequency in a range of about 1 kHz to about 1 MHz.

6. The sensor of claim 4, wherein the AC voltage has an amplitude in a range of about 100 millivolts to about 3 volts.

7. The sensor of claim 1, wherein the electrical property comprises a DC electrical resistance.

8. The sensor of claim 1, wherein the substrate comprises a semiconductor,

9. The sensor of claim 8, wherein the semiconductor comprises silicon.

10. The sensor of claim 1, wherein the graphene layer comprises a plurality of graphene nano-flakes.

11. A method of detecting a prostate-specific biomarker in a sample, comprising:
applying a sample to a graphene layer functionalized with an antibody exhibiting specific binding to a prostate-specific biomarker, measuring at least one electrical property of the antibody-functionalized graphene layer, and
using the measured electrical property to determine whether the prostate-specific biomarker is present in the sample.

12. The method of claim 11, further comprising quantifying the prostate-specific biomarker in said sample.

13. The method of claim 11, wherein the sample comprises a biological sample.

14. The method of claim 13, wherein the biological sample comprises any of blood, urine, and semen.

15. A sensor for detecting a prostate-specific biomarker in a sample, comprising:
a graphdiyne layer deposited on an underlying substrate,
a plurality of antibodies coupled to the graphdiyne layer to generate antibody-functionalized graphdiyne layer, wherein the antibodies exhibit specific binding to a prostate-specific biomarker, and
a plurality of electrical conductors electrically coupled to the antibody-functionalized graphdiyne layer for measuring an electrical property thereof.

16. The sensor of claim 15, wherein the graphdiyne layer comprises a plurality of graphdiyne flakes.

17. The sensor of claim 15, wherein the prostate-specific biomarker comprises a prostate-specific antigen (PSA).
